(19) United States
(12) Patent Application Publication
(10) Pub. No.: US 2006/0177376 A1
(43) Pub. Date: Aug. 10, 2006

(54) Stabilized and chemically functionalized nanoparticles

(75) Inventors: Donald A. Tomalia, Midland, MI (US); Boahua Huang, Mt. Pleasant, MI (US)

Correspondence Address: TECHNOLOGY LAW, PLLC, 3595 N. SUNSET WAY, SANFORD, MI, 48657, US

(73) Assignee: Dendritic Nanotechnologies, Inc., Mt. Pleasant, MI (US)

(21) Appl. No.: 10/565,478

(22) PCT Filed: Jul. 21, 2004
(86) PCT No.: PCT/US04/23483

§ 371 Date: Jan. 20, 2006

Related U.S. Application Data
(60) Provisional application No. 60/488,909, filed on Jul. 21, 2003.

(51) Int. Cl. A61F 2/00

(52) U.S. Cl. 424/9.3

(57) ABSTRACT

Dendronization of nano-scale surfaces with focal point reactive dendrons to produce stabilized chemically functionalized nano-particles having quantum dot dimensions.

This application claims priority from U.S. Provisional Application 60/488,909, filed on Jul. 21, 2003.

### BACKGROUND OF THE INVENTION

This invention deals with dendronization of nano-scale surfaces with focal point reactive dendrons to produce stabilized chemically functionalized nano-particles having quantum dot dimensions.

The design of nanoscale molecular architecture, using the convergent polymerization technique, or “the bottom up approach” has offered a wide range of possibilities for creating new optoelectronic materials. Such an approach requires systematic and rigorous control over size, shape, and surface chemistry in order to capture critical nano-properties anticipated from these important targets. Dendrons and dendrimers are precise quantized, three-dimensional nanostructures that offer such control and are of keen interest to both nano-scientists as building blocks and to polymer scientists due to their unique, architecturally driven, macromolecular properties. Architecturally, dendrons and dendrimers are core-shell nanostructures consisting of (a) core, (b) interior branch cells and (c) an exponential number of functional surface groups (Z), that amplify as a function of the expression: Z=NₒN_{b}^{G}; where G=generation and Nₒ, N_{b} are core and branch cell multiplicities, respectively. All of the above parameters may be combinatorially tuned to fit many important biomedical and optoelectronic applications. Dendronization is a widely accepted term that describes either the covalent or supramolecular attachment of dendrons to non-dendritic properties. By definition, a dendron has a core multiplicity (Nₒ) of one, therefore amplification of surface (terminal) groups, (Z) is solely dependent upon the branch cell multiplicity (N_{b}) and the generation level, (G) of the dendron.

Semiconductor, metal, and metal salt nanocrystallites (quantum dots) whose radii are smaller than the bulk exciton Bohr radius constitute a class of materials intermediate between molecular and bulk forms of matter. Quantum confinement of both the electron and hole in all three dimensions leads to an increase in the effective band gap of the material with decreasing crystallite size. Consequently, both the optical absorption and emission of quantum dots shift to the blue (higher energies) as the size of the dots get smaller.

Bawendi and co-workers have described a method of preparing monodisperse semiconductor, metal, and metal salt nanocrystallites by pyrolysis of organometallic reagents injected into a hot coordinating solvent. See J. Am. Chem. Soc., 115:8706 (1993). This permits temporally discrete nucleation and results in the controlled growth of macroscopic quantities of nanocrystallites. Size selective precipitation of the crystallites from the growth solution provides crystallites with narrow size distributions. The narrow size distribution of the quantum dots allows the possibility of light emission in very narrow spectral widths.

Although the Bawendi semiconductor nanocrystallites exhibit near monodispersity, and hence, high color selectivity, the luminescence properties of the crystallites are poor. Such crystallites exhibit low photoluminescent yield, that is, the light emitted upon irradiation is of low intensity. This is due to energy levels at the surface of the crystallite that lie within the energetically forbidden gap of the bulk interior. These surface energy states act as traps for electrons and holes that degrade the luminescence properties of the material.

Thus, in an effort to improve photoluminescent yield of the quantum dots, the nanocrystallite surfaces have been passivated by reaction of the surface atoms of the quantum dots with organic passivating ligands, so as to eliminate forbidden energy levels. Such passivation produces an atomically abrupt increase in the chemical potential at the interface of the semiconductor and passivating layer.

Bawendi, Supra, described CdSe nanocrystallites capped with organic moieties such as tri-n-octyl phosphine (TOP) and tri-n-octyl phosphine oxide (TOPO) with quantum yields of around 5 to 10%. Passivation of quantum dots using inorganic materials also has been reported. Particles passivated with an inorganic coating are more robust than organically passivated dots and have greater tolerance to processing conditions necessary for their incorporation into devices.

Such materials are CdS-capped CdSe and CdSe-capped CdS; ZnS grown on CdS; ZnS on CdSe and the inverse structure, and SiO₂ on Si. These materials have been reported as exhibiting very low quantum efficiency and hence are not usually commercially useful in light emitting applications.

In U.S. Pat. No. 6,322,901 to Bawendi, et al, that issued on Nov. 27, 2001, there is disclosed the preparation of coated nanocrystals capable of light emission that include a substantially monodisperse nanoparticle selected from the group consisting of CdX, where X═S, Se, Te and an overcoating of ZnY, where Y═S, Se, uniformly deposited thereon. The coated nanoparticles are characterized, in that, when irradiated, the particles exhibit photoluminescence in a narrow spectral range of no greater than about 60 nm, and most preferably 40 nm, at full width half max (FWHM).

Thus, there remains a need for semiconductor, metal, and metal salt, nanocrystallites capable of light emission with high quantum efficiencies throughout the visible spectrum that possess a narrow particle size and hence have narrow photoluminescence spectral range.

Elsewhere, G. Schmid, in “In Progressive Colloid Polymer Sciences”, 111, pp. 52 to 57, (1998), discloses the properties of small, protected clusters of metal atoms with dimensions of between 1 nanometer and 15 nanometers. Schmid labeled these particles “quantum dots” or “artificial atoms”, and defined them as metal particles/clusters that have been reduced to a size comparable to the de Broglie wave length of an electron (d) leading to the formation of stationary electronic waves with discrete energy levels. In that particle size range, quantum confinement effects are observed. The smaller the cluster size the more dramatic the effect at room temperature. For example, the current/voltage (I/V) characteristics for a 17 nanometer palladium cluster shows a temperature dependent effect.

At room temperature, this cluster behaves as a metallic, however, at 4.2K, when the electrostatic energy exceeds the thermal energy of the electron, there is a pronounced Coulomb gap that indicates energy quantization. The smaller the particle the higher the “quantum confinement effect” at room temperature. The inclusion of electrons in a quantum dot that is isolated from others by a non-conductive material, i.e., a ligand shell, is possible if the particle diameter corresponds to λ/2 wherein λ is the de Broglie wavelength.

It is very import to sheath and protect quantum dots with generally organic compositions that function as both a barrier to oxidation, as well as direct metal-to-metal particle contact, that can lead to aggregation and precipitation. Furthermore, it is important that such organic sheathing should provide suitable solubility parameters for dissolving these quantum dots. It is also important to provide desirable chemical functionality to allow the quantum dots to be combined to function as surface reactive composites in a variety of nano-devices. Generally mercaptans or phosphine-terminated alkyl hydrocarbons have been used as such protective coatings.

Thus, there remains a need for semiconductor, metal, and metal salt, nanocrystallites capable of light emission with high quantum efficiencies throughout the visible spectrum that possess a narrow particle size and hence have narrow photoluminescence spectral range.

### BRIEF DESCRIPTION OF THE DRAWINGS

FIG. 1 illustrates the synthesis and surface modification of Generation 1 and Generation 2 dendrimers and the reduction of the Generation 2 material to the thio bearing, single focal point dendron, beginning with a cystamine core, ORGANIC dendrimer.

FIG. 2 illustrates the formation of a dendronized nanoparticle wherein the core material for the nanoparticle is gold and the dendron is that generated in the reaction of FIG. 1.

FIG. 3 is a schematic of ligand exchange of nanoparticle with dendron phosphine oxide compounds.

FIG. 4 is a detailed synthesis of a dendritic phosphine ligand as is set forth in example 2, First Part.

FIG. 5 is a detailed synthesis of a dendritic phosphine ligand as is set forth in example 2, Second Part.

FIG. 6 is a detailed synthesis of a dendritic phosphine ligand as is set forth in example 2, Third Part.

FIG. 7 is a detailed synthesis of a dendritic phosphine ligand as is set forth in example 2, Fourth Part.

FIG. 8 is a drawing of a dendron illustrated as a cone.

FIG. 9 is a chemical formula illustrating the makeup of the cone of FIG. 8.

FIG. 10 is a drawing of a gold nanoparticle considered as being spherical.

FIG. 11 is an illustration of a nanoparticle (a) having cone-shaped dendrons on the surface.

FIG. 12 is an illustration of a nanoparticle (b) having cone-shaped dendrons on the surface.

FIG. 13 is an absorption spectra of Gold-Generation 1 in water.

FIG. 14 is an absorption spectra of Gold-Generation 2 in water.

FIG. 15 is an absorption spectra of Gold-Generation 3 in water.

FIG. 16 is an absorption spectra of CdSe/CdS core-shell quantum dots stabilized by citrate (a), Generation −2 polyether phosphine ligand (b) made by this invention, and Generation −2 PAMAM sulfhydryl ligand made by this invention.

FIG. 17 is a luminescence spectra CdSe/CdS core-shell quantum dots stabilized by citrate (a), Generation −2 polyether phosphine ligand (b) made by this invention, and Generation −2 PAMAM sulfhydryl ligand made by this invention.

FIG. 18 is a schematic of the synthesis of the poly ether dendron with a phosphine focal point.

### THE INVENTION

This invention deals with dendronization of nano-scale surfaces with focal point reactive dendrons to produce stabilized chemically functionalized semiconductor, metal, and metal salt, nano-particles having nano/micron scale dimensions in the range of 1 to 10,000 nanometers. The inventors herein have discovered that dendrons having certain characteristics can provide the sheathing required to protect the nano-scale surfaces and provide materials having a variety of properties. What is meant by “dendrons” in this invention are those organic dendrons that are prepared from organic compositions.

One of the means for providing fragments is to provide the appropriate dendrimer. The appropriate dendrimer for producing the dendron fragments required for the sheathing can be, for example, based on disulfide type core dendrimers or dendritic polymers that will be set forth infra. An example of such dendrimers can be found in U.S. Pat. No. 6,020,457 that issued to Klimash, et. al. that deals with disulfide-containing dendritic polymers. Recent access to important single site, thio core, functionalized organic dendrons now allows the direct dendronization of a wide variety of nano-substrates. This U.S. patent is incorporated herein by reference for what it teaches about the preparation of the disulfide-containing dendritic polymers and their properties.

In the past, nanoparticles (colloids) have been stabilized with a variety of surfactants and used to label biomolecules such as proteins, peptides, carbohydrates, lipids and DNA due to their visually dense properties as electron microscopy labels or nanoscale plasmon properties. There are many traditional methods for synthesizing nanoparticles, ranging in size from 2 to 30 nm, including the classical citrate method.

However, products obtained by these techniques have several deficiencies. Most notably, the nanoparticles are generally prone to aggregation if the reaction conditions are not carefully controlled and the versatile introduction of tunable surface chemistry is difficult at best. For these reasons, new routes for the preparation of stable chemically functionalized metal cluster nanoparticles are of keen interest.

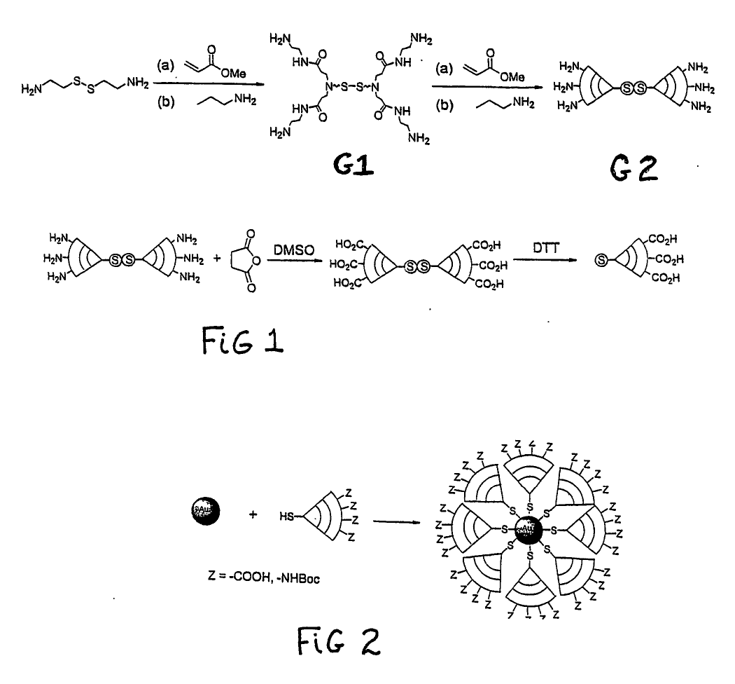

Recent access to important single site, thio core, and functionalized PAMAM dendrons now allows the direct dendronization of a wide variety of nano-substrates. The synthesis and surface modification of Generation 1 and Generation 2; cystamine core, PAMAM dendrimers is shown in FIG. 1 and the use of the dendrimers to form the dendron is shown in FIG. 2. The particle size is from 1 nanometer to 100 nanometers, and in this case, by way of example, gold is shown in FIG. 2.

In a further embodiment, this invention deals with preliminary luminescence properties of dendronized metal nanoparticles manufactured from CdSe/CdS core shell quantum dots using single site, thiol functionalized PAMA dendrons.

It is contemplated within the scope of this invention to include dendrimers other than disulfide type core dendrimers, such as, for example, those containing phosphorus atoms.

Contemplated within the scope of this invention are functional groups on the surface of the dendrimers/dendrons that are certain hydrophilic, hydrophobic, reactive or passive groups that include, by way of example such groups as: hydroxyl, amino, carboxylic, sulfonic, sulfonato, mercapto, amido, phosphino, —NH—COPh, —COONa, alkyl, aryl, ester, heterocylic, alkynyl, alkenyl, and the like. The generation level of the dendrimer can range from about zero to ten.

The metal cores can be any semiconductor, metal or metal salt that will react with or adsorb the functional group of the dendrons, for example, but not limited to Au, Ag, Cu, Pt, Pd, Fe, Co, Ni, Zn, Cd, or their alloys; magnetic compositions such as Fe compounds, Fe₂O₃, Ni, and the like, metal salt and oxides/sulfides/selenides such as CdSe, CdS, CdSe/CdS, CdSe/ZnS, CdTe, CdTe/CdS, CdTe/ZnS, and such materials that have been passivated.

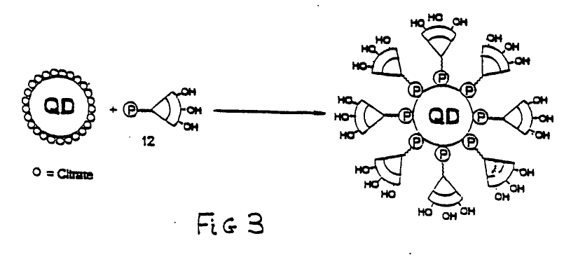

Contemplated within the scope of this invention are the above-mentioned materials wherein the core is bonded to the dendritic material with phosphorus-containing materials, such as phosphines, for example, aryl, alkyl and mixed aryl/alkyl phosphines and aryl, alkyl and mixed aryl/alkyl phosphine oxides. The phosphines are those having the formula 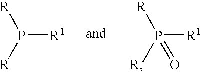
wherein each R is independently selected from alkyl radicals having 1 to 4 carbon atoms and aryl groups, and R¹ is a functionally reactive connector group, for example a benzoic acid radical. Such materials are bound to the dendritic material and then, they bind through the phosphine to the quantum dot. (See FIG. 3). The preferred materials are the aryl phosphines. These materials are stable in air and are less toxic than alkyl phosphines. The aryl groups that are UV active at 200 nm, will not block any photoluminescence, that is above 500 nm. Most importantly, phosphine passivation set forth above many quench the photoluminescence that is essential for bio labeling.

Such materials can be illustrated by reference to FIGS. 4 through 7, wherein there is shown the synthesis of dendritic phosphine ligands using diphenylphosphino)-benzoic acid.

Encapsulating quantum dots and their initial ligands with polymers can preserve them, but generally it adds a large volume to the quantum dots resulting in a final size that can be much bigger than desired. As set forth above, quantum dots have been stabilized using phosphines, but no polymer had been added. In this invention, preferred are novel dendritic polyether compounds containing aryl phosphine at the focal point to stabilize the quantum dots.

As indicated Supra, dendrimers are well defined and highly branched macromolecules, and are of great interest as new materials for application in many areas. Such dendrimers contain an initiator core, interior branching units, and a number of functional surface groups. The structure of the dendrimer is ideal to stabilize quantum dots because their steric crowding characteristics may provide a closely packed but thin ligand shell that may be as efficient as a shell formed by the ligands with a long and floppy single chain, or a polymer shell. Importantly, the steric crowding of a dendron is very ideal for filling the spherical ligand layer because the dendron ligand can naturally pack in a cone shape on the surface of the nanocrystals (see FIGS. 11 and 12).

In estimating the theoretical number of dendrons attached to gold nanoparticles, for example, one has to assume that the nanoparticles are spherical (see FIG. 10, wherein R=radius) and the dendron moieties are cones (see FIG. 8, wherein r=radius and h=height). Also note that the cone shape is recognizable in the chemical formula that is shown in FIG. 9. The maximum number (Nₘₐₓ) of dendrons could be attached to the nanoparticle is describe by the equation

$$( {N}_{max} ) = \frac{2 {Π ( R + h )}^{2}}{√ 3 {r}^{2}}$$

Considering each cone is solid, the interior space of each conjugated product for guest molecules to be encapsulated can be calculated using the equation

$$V = \frac{4}{3} {Π ( R + h )}^{3} - \frac{4}{3} Π {R}^{3} - {N}_{max} \frac{1}{3} Π {R}^{2} h$$

Using the above equations, the maximum number of dendrons that can be attached to the nanoparticles and the interior space of each complex are found in the Table below.

TABLE

| | G0 | | G1 | | G2 | | G3 | |
|---|---|---|---|---|---|---|---|---|
| | r = 0.55 nm^{a} | | r = 0.85 nm | | r = 1.55 nm | | r = 1.7 nm | |
| Particle | h = 1.2 nm | | h = 2.0 nm | | h = 2.7 nm | | h = 3.5 nm | |
| size | Nₘₐₓ/ | | Nₘₐₓ/ | | Nₘₐₓ/ | | Nₘₐₓ/ | |
| 2 R (nm) | V (nm3)^{b} | | V (nm³) | | V (nm³) | | V (nm³) | |
| 2.5 | 72 | (26) | 53 | (56) | 24 | (87) | 28 | (144) |
| 5.0 | 164 | (84) | 101 | (1630 | 41 | (245) | 45 | (363) |
| 10.0 | 469 | (300) | 246 | (541) | 89 | (784) | 91 | (1085) |

^{a}= sizes of dendrons calculated using MM2 force field to minimize energy.
^{b}= considering each cone is solid, actually each dendron has interior space, especially at higher generations.

The inter- and intramolecular chain tangling of the dendron with relatively flexible branches may further slow the diffusion of small molecules or ions from the bulk solution into the intertice between the nanocrystal and its ligand.

The units of ethylene glycols between the focal point and the dendritic structure are for enhancement of aqueous solubility. For purposes of this invention, the number of ethylene groups between the focal point and the dendritic structure can be from 1 to 10. Surface groups for these materials are those set forth Supra, such as certain hydrophilic, hydrophobic, reactive or passive groups that include, by way of example such groups as: hydroxyl, amino, carboxylic, sulfonic, sulfonato, mercapto, amido, phosphino, —NH—COPh, —COONa, alkyl, aryl, ester, heterocylic, alkynyl, alkenyl, and the like. The generation level of the dendrimer can range from zero to ten.

FIG. 4 shows a schematic of the theory of the structure and placement of dendrimers on the quantum dot surface. What is illustrated is the estimate of theoretical number of dendrons that are attachable to gold nanoparticles. Cystamine core PAMAM dendrimers were reduced in water to dendrons with sulfhydryl reactive points. Then these solutions were added to a as-synthesized gold colloidal solution. The schematic synthesis is set forth in FIGS. 1 and 2.

The advantages of the materials of the instant invention are many and include the provision of denser, thicker insulating type sheathing than would be expected with traditional sheathing. This sheathing better protects the quantum dots advantageously against oxidation, hydrolysis, thermal, chemical or photochemical attacks.

The ability to functionalize this unique dendritic sheathing with the unlimited examples of dendritic polymeric surfaces functionality allows one to produce very versatile, polyvalent functional surfaces groups on a side variety of metallic quantum dots that includes both metals as well as metal salt or derivatives that may exhibit a wide variety of properties, such as semi-conductivity, paramagnetic, magnetic, fluorescing, electrotumescent, and the like.

The resulting core-shell type structures are novel and useful as biologically active materials, genetic materials, or biologically active materials for use as vaccines and for use as biomedical tags, as components in light emitting diode devices, such as LED's, for diagnostics, as nanosensors, and in nano-arrays for DNA and RNA or protein applications, chelators, photon absorption, energy absorbing, or energy emitting, as a signal generator for diagnostics, and thus these materials may contain radioactive materials. For example, when iron is the core metal, these materials are MRI agents and when gold or other dense elements are the core metal, they can be used as projectiles for gene guns.

The polyvalent surfaces of these quantum dot-core-dendritic shell structures are used for the targeted delivery with antibody attachments, receptor directed targeting groups such as folic, biotin/avidin, and the like.

The interior of the structures can be made catalytic and which can avoid poisoning entities but are accessible to an entity that is catalytically converted to a desirable product. These materials can also be made to contain drugs, pharmaceuticals, fragrances, and can be used as agricultural chemicals, or encapsulants for controlled release applications, or for gene gun applications.

These metallic domains can be provided in a variety of shapes including spherical, ellipsoidal, rod or rod-like, cylindrical, branched, for example in a (Y) or (+) shape, or can be comb-shaped, for example (+++++), and may be 2-dimentional or flat with irregular shapes and are not limited by geometrical regularity.

One preference for materials of this invention are poly(amidoamine) (PAMAM) dendrimers that can be reduced at the cystamine core to produce mercapto-functional dendrons. The precursor dendrimer can be derived from different generations with different surfaces.

With reference to FIG. 1, there is shown the formation of the functionalized dendrimer using a disulfide linkage. Two dendrons are attached together by a disulfide group to provide the dendrimer. Upon reduction, the disulfide group splits into mercapto-functional dendrons. Note that other hetero atoms can be substituted for the sulfur in the molecules.

### EXAMPLES

### Example 1

There was provided a generation one, cystamine core, succinic acid surface dendrimer (59 mg, molecular weight of 2323, 0.0250 mmol) that was dissolved in DI water (0.5 ml.) that had been purged with nitrogen for 15 minutes. Then DTT (3.3 mg, 0.9 eq./dendrimer) was added. The mixture was stirred at room temperature under nitrogen overnight (approximately 16 hours).

Three solutions were prepared: (1.) 0.2 M potassium carbonate using 2.764 gms. dissolved in 100 ml of DI water; (2.) 4% HAuCl₄ using 82.1 mg of HAuCl₄.3H₂O dissolved in 1.70 ml. of DI water, and (3.) 0.5 mg/ml NaBH₄ using 4.0 mg of NaBH₄ dissolved in 8.0 ml of DI water.

One hundred ml of DI water was put into a 250 ml round-bottomed flask with a magnetic stirring bar. The flask was cooled to 0° C. with an ice-water bath. Five hundred microliters of the potassium carbonate solution and 375 microliters of gold solution was added and mixed well. Then 5 ml of the sodium borohydrate solution was added ml at a time, with rapid stirring. A color change from bluish-purple to reddish-orange was noted as the addition took place. The reaction was stirred for 5 minutes at 0° C.

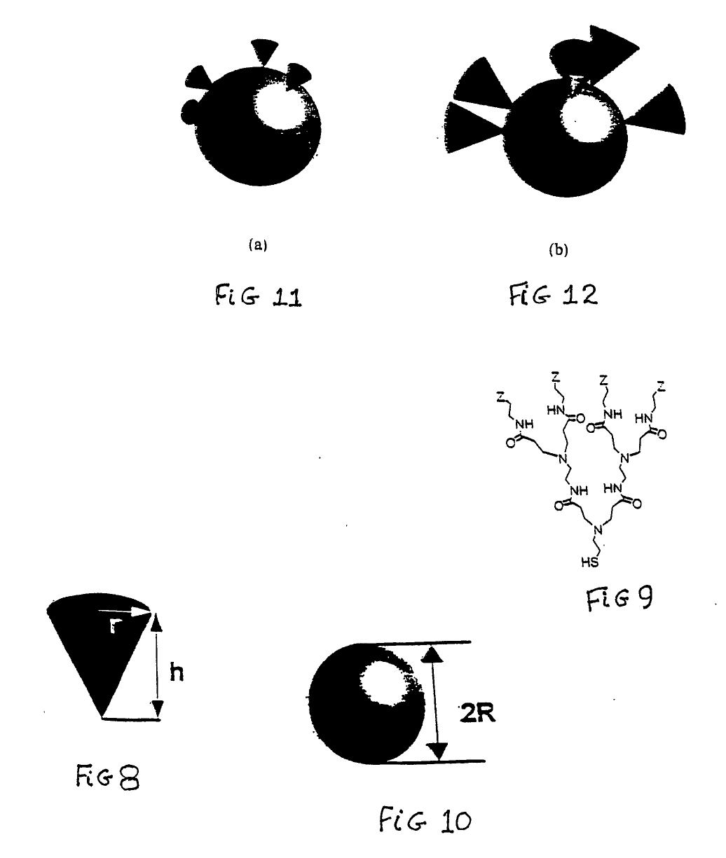

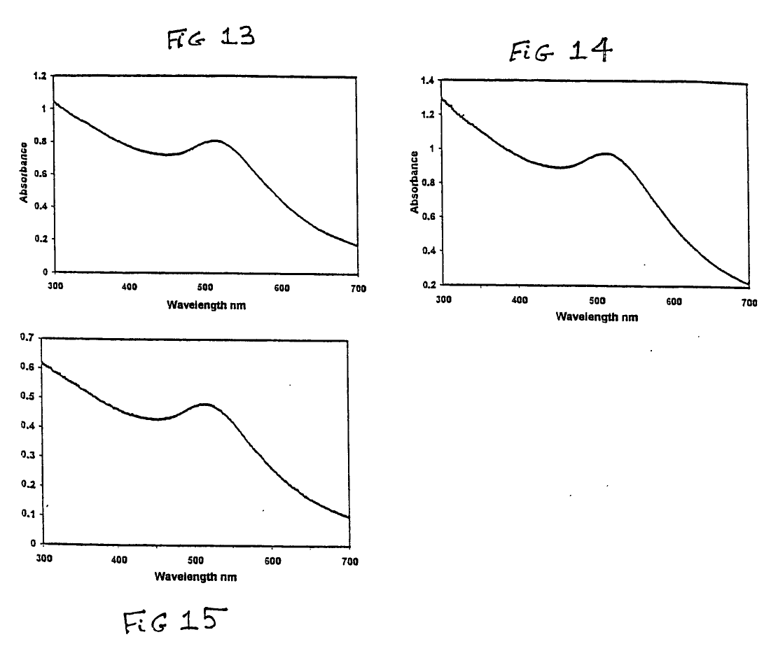

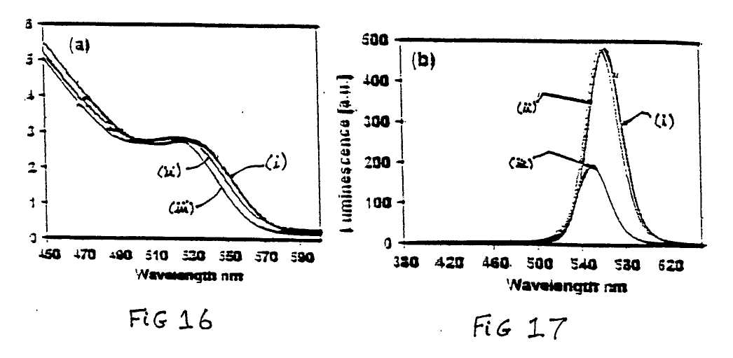

Then dendrimer solution was then added in 0.25 ml increments. The color of the reaction changed from reddish-orange to bluish-purple. The reaction was stirred at 0° C. for 10 minutes and then the ice water bath was removed and the reaction was allowed to warm to room temperature while stirring for 24 hours in the absence of light. Water was removed under reduced pressure (29.5 in Hg at 25° C.) water bath temperature. Then 4 ml of methanol were added to the residue. The black precipitate was removed to a small vial with methanol and it was let stand for 15 hours at −15° C. The yield was 8.0 mg. The absorption spectra for the compounds Au-G1, Au-G2, and Au-G3, are found in FIGS. 13, 14, and 15, respectfully.

### Example 2

(First Part)—With reference to FIG. 4, the hydroxyl in 1-methyl-4-(hydroxymethyl)-2,6,7-trioxabicyclo-{2.2.2}-octane (MHTBO 1), was protected with the benzyl group. Then the hydrolysis of the orthoester using a trace of concentrated hydrochloric acid in methanol exposes three hydroxyl groups to give compound 3. The tosylation of 3 gives compound 4 in high yields. Then, there are several problems. The attempt to react the tosylated product with alkoxide of 1 directly without being converted to abromide fails because of the steric hindrance. 2. During the purification of the product of the previous reaction, the orthoester was proved not stable to aqueous work up and partially hydrolyzed on silica gel. 3. The reaction of deprotecting the benzyl group is very slow probably because the steric hindrance of the other three bulky branches; and in the meantime, the orthoester can be cleaved partially during the catalytic hydrogenation.

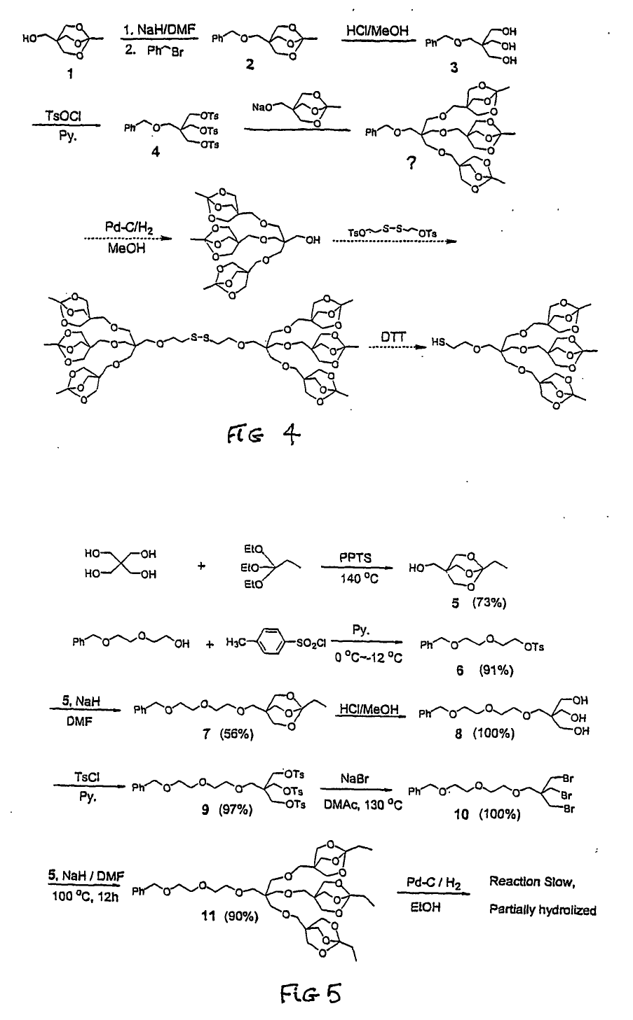

(Second Part)—With reference to FIG. 5, pentaerythritol was protected with trimethyl orthopriopionate to give 1-ethyl-4-(hydroxymethyl)-2,6,7-trioxabicyclo-{2.2.2}-octane in moderate yield, the desired product was distilled under high vacuum (compound 5). This compound was used as a branching unit in a later generation growth. Di(ethylene glycol)benzyl ether was tosylated to give the compound 6. Without bromination, compound 6 was reacted with the alkoxide of 5 to give generation zero polyether dendron, compound 7. Compound 7 was partially hydrolyzed when purified using silica gel chromatography. Partially hydrolyzed compound and 7 could be totally hydrolyzed by trace concentrate hydrochloric acid in methanol, to give 8 in quantitative yield. The tosylation of 8 was performed in pyridine, and 9 was purified by chromatography in high yield. In order to avoid the defection which generating growth, the toslylated compound 9 was converted to bromide 10, quantitatively. The reaction was carried out in dimethyl acetamide at 130° for 2.5 hours. The product was used for the next step without any further purification. Then 10 was reacted with the alkoxide of 5 (1.2 eq./bromide), to give the first generation polyether dendron 11. The reaction was carried out at 100° for 12 hours. TLC was used to monitor the reaction. TLC showed that the first branch was substituted instantly, the second one and the third one were much slower. The reaction was clean, taken up with dichloromethane and washed with sodium bicarbonate solution. NMR showed this work up procedure as efficient, and no further purification was needed. An attempt to deprotect the benzyl group at 1 atmosphere was then performed. The reaction was very slow (2 days, only about half of the starting material was consumed as indicated by TLC. Furthermore, there were several more new spots on TLC, indicating that the orthoester had been partially hydrolyzed in these conditions which indicates that ethyl orthoester was not more stable than the methyl analog.

(Third Part)—With reference to FIG. 6, a second attempt was made to find more stable protecting groups for the three hydroxyls on the surface of the branching unit other than orthoesters. The protecting group must be stable to aqueous work up and silica gel columns, and should be stable to palladium-carbon catalytic hydrogenation. Methoxy methyl (MOM) ether and 2 methoxy ethoxymethyl HEM) ether are well used protecting groups for hydroxyl. Compound 3 was treated with MOM chloride or MEM chloride to give the corresponding MOM or MEM protected products 1 and 13 in moderate yield. These two compounds could be purified by silica gel chromatography. Then deprotection of benzyl groups by catalytic hydrogenation at 55 psi gave the new branching units 14 and 15. The rate of hydrogenation of 12 and 13 were quite different, 12 being much slower (5 days) than 13 (2 days).

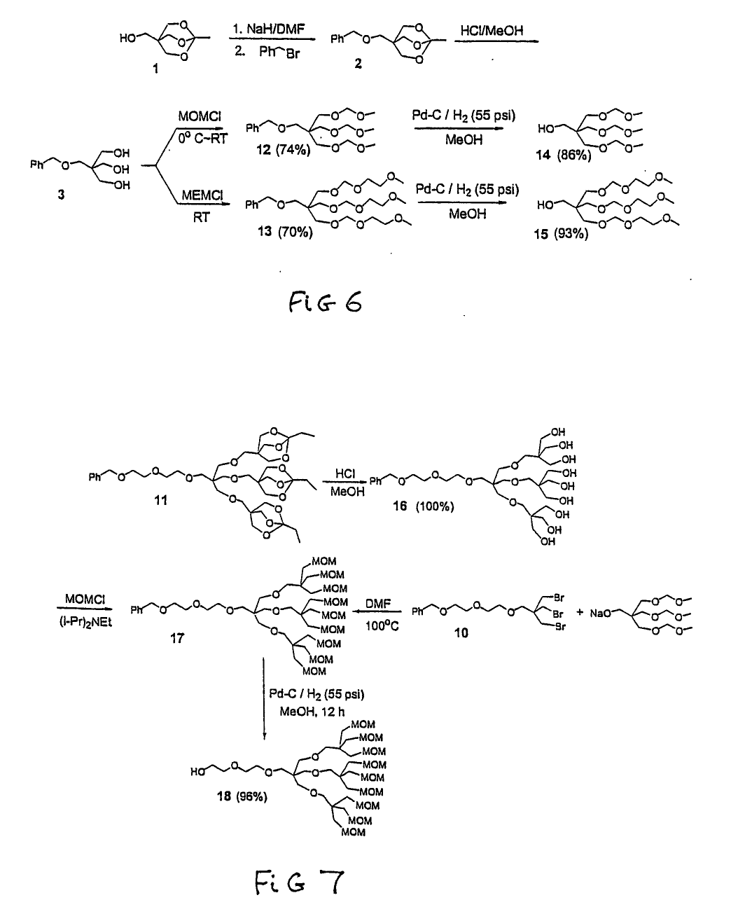

(Fourth Part)—Thereafter, with reference to FIG. 7, generation 1 polyether dendron is synthesized in two ways. The hydrolyzation of Bn-G1-(ethyl orthoester)₃ 11 gave Bn-G1-(OH)₉ polyether dendron 16 in quantitative yield. Then all of the 9 hydroxyl groups were protected by MOM to give Bn-G1-(MOM)₉ compound 17. Compound 17 can also be synthesized by the reaction of Bn-G0-Br₃ 10 with the alkoxide of the new branching unit 14 at 100° C. for 12 hours in DMP. The yield of 14 is not high in both procedures, probably due to the adsorption on silica gel during purification. The catalytic hydrogenation to deprotect the benzyl was carried out in methanol, and reaction time was 12 hours with almost quantitative yield. The product Ho-G1-(MOM)₉ 18 is very clean.

This structure contains one hydroxyl functional group at the focal point, and 9 protected hydroxyl groups on the surface. The one hydroxyl at the focal point can be converted to sulfhydryl, phosphine or other functional group for attaching purposes. Deprotection of the hydroxyl groups can make the dendron soluble in aqueous solution, or the hydroxyl can be transferred to other functional groups to get the desired properties. Examples 3 to 19 deal with the details of the experiments

### Example 3

In 25 mL of anhydrous DMF was dissolved 1-methyl-4-(benzyloxymethyl)-2,6,7-trioxabicyclo-{2.2.2}-octane 2 HBO) 1 (5.0 g, 31.2 mmol)and was slowly added to a suspension of NaH (840 mg, 35 mmol; 1.4 g of 60% NaH dispensed in mineral oil and washed with hexane) in 25 Ml of DMF. The mixture was stirred for 45 min. then 4.1 Ml (5.9 g, 34.5 mmol) of benzyl bromide was added dropwise. Then the reaction was stirred at room temperature over night. Solvent was removed by rotary evaporation until 10 ml of DMF was left. The residue was slowly poured into 200 mL of DI water. A pale white solid precipitated out and was filtered to give 2 (6.64 g, 85.4%). This compound was used for the next step without further purification.

### Example 4

### Preparation of Bn-G0-(OH)₃ (3)

Compound 2 (6.64 g, 29.3 mmol) was dissolved in 70 mL of methanol. Then 1 mL of concentrated HCl was added and the mixture was heated to 70° C. for 2 hours. TLC showed that all starting material was consumed. Solvent was removed and the residue was put on high vacuum for over night to give 3 as a slightly yellow oil (6.05 g, 100%.).

### Example 5

### Preparation of Bn-G0-(Ots)₃ (4)

Compound 3 (4.69 g, 20.7 mmol) was dissolved in 30 mL pyridine and was cooled to 0° C. Then tosyl chloride (13.02 g, 68.3 mmol) was added and the reaction was allowed to stand at −12° C. for 48 hours. Then solvent was removed and the residue was washed with 10% HCl and brine. Organic layers were combined and after evaporate of solvent gave 4. It was used without further purification.

### Example 6

Compound 1-ethyl-4-hydroxymethyl)-2,6,7-trioxabicyclo0{2.2.2}-octant (EHTBO, 5), pentaerythritol (27.2 g, 0.2 mmol), trimethyl orthopriopionate (35.3 g, 0.2 mmol) and pyridinium p-toluenesulfonate (PPTS, 1.0 g, 0.004 mol) were put into a 250 mL round bottomed flask, attached to a Dean-Stark trap fitted with a reflux condenser. The mixture was heated at 140° C. with periodic shaking, under nitrogen. The solid in the reaction disappeared after 1 hour heating and the mixture became homogeneous. After 3.5 hours heating, the reaction released almost quantitative amounts of ethanol (32 mL, theoretical). The nitrogen line was replaced with a house vacuum line to remove trace of ethanol. The residue was distilled under vacuum at 140 to 150° C. to give the product as a colorless oil which solidified in freezer as a white crystal (23 g, 73%) ¹H NMR(DMSO-d⁶, 300 MHz)δ: 0.8(t, J=7.5 Hz, 3H),1.54(q, J=7.5 Hz, 2H), 3.21(d, J=5.7 Hz, 2H), 3.85(s, 6H), 4.75(t, J=5.4 Hz, 1H); ¹³CNMR (DMSO-d⁶, 100 MHz) δ: 7.67, 29.63, 35.41, 59.53, 68.89, 108.93 ppm.

### Example 7

### Tosylation of di(ethylene glycol)benzyl ether (6)

Di(ethylene glycol) benzyl ether (5.016 g, 25.56 mmol) was added and the reaction was put in a −12° C. freezer overnight. The pyridine was removed and the residue was taken up in dichloromethane and washed with diluted HCl and brine. After the evaporation of the solvent 6 was obtained as a colorless oil (8.1 g, 91.3%). ¹H NMR (CDCl3, 300 MHz δ:2.39(s, 1H), 3.52-3.61(m, 4H), 3.66(t, J=7.5 Hz, 2H), 4.51(s, 2H), 7.22-7.34 (m, &H), 7.77 (m, 2H); ¹³C NMR (CDCl₃, 100 MHz) δ:21.35, 68.40, 69.11, 69.14, 70.51, 72.97, 127.38, 127.69, 128.14, 129.6, 132.74, 137.95, 144.58 ppm

### Example 8

### Preparation of Bn-G0-(ethyl orthoester) (7)

EHTBO 5 (3.83 g, 22 mmol) was dissolved in 10 mL anhydrous DMF and slowly added to a suspension of NaH (581 mg, 24.2 mmol); 968 mg of 60% NaH dispensed in mineral oil that was washed with hexane) in 10 mL of DMF. The mixture was stirred for 45 min. Then a solution of 6 (7.0 g, 20 mmol) in DMF (5 mL) was added dropwise. Then the reaction was stirred at room temperature over night. Solvent was removed using a rotary evaporator and the residue was taken up in 30 mL dichloromethane, and washed with 5% NaHCO₃. After removal of solvent, the product was purified by silica gel chromatography (ethyl acetate: hexane=2:1) to give 7 (3.5g, 50%).

### Example 9

### Preparation of Bn-G0-(OH)₃ (8)

Bn-G0-(ethyl orthoester) 7 (2.42 g, 6.88 mmol) was dissolved in 17 mL methanol. Then 0.5 mL concentrated HCl was added and the reaction was heated to 70° C. for 2 hours. After solvent was removed, the residue was put on high vacuum over night to give Bn-G0-(OH)₃ 8 as a slightly yellow oil (2.159 g, 100%). ¹H NMR (CDCl₃, 500 MHz) δ: 3.48(s, 2H), 3.58-3.65(m, 14H), 4.27(s, 3H), 4.54(s, 2H), 7.26-733(5H); ¹³C NMR (CDCl₃ 125 MHz) δ: 21.55, 43.68, 66.77, 67.21, 69.32, 69.99, 70.49, 70.73, 73.10, 127.5, 127.64, 127.83, 128.26, 129.93, 131,84, 138.14, 145.18 ppm

### Example 10

### Preparation of Bn-G0-(Br)₃ (10)

Bn-G0-Ots)₃ 9 (1.12 g, 1.44 mmol) was dissolved in dimethyl acetamide (10 mL). Then sodium bromide (1.11 g, 10.9 mmol) was added and the reaction was heated to 130° C. for 2.5 hours. Then solvent was removed and the residue was taken up in dichloromethane (20 mL). The organic layer was washed with water (3×20 mL) and brine. After the evaporation of solvent there was Gn-G0-(Br)₃ as a colorless oil (690 mg, 96%). ¹H NMR (CDCl₃, 500 MHz) δ:3.51 (s, 6H), 3.52(s, 2H), 360.3.67(m, 8H), 4.56(s,2H), 7.24-7.34(m, 5H); ¹³C NMR (DCCl₃, 125 MHz) δ: 34.84, 43.70, 69.43, 69.77, 70.34, 70.58, 70.93, 73.19, 127.53, 127.64, 128.29, 138.18 ppm

### Example 11

### Preparation of Bn-G1-(ethyl orthoester)₃ (11)

EHTBO 5 (825 mg, 4.71 mmol) was added slowly to a suspension of NaH (133 mg, 5.54 mmol, 218 mg 60% NaH in mineral oil) in 2 mL anhydrous DMP. The mixture was stirred for 45 min. until all of the gas was released. Then a solution of Bn-G0-(Br)₃ 10 (586 mg, 1.167 mmol) in 2 mL DMF was added to the alkonide solution dropwise. After the addition, the reaction was heated to 100° C. for 10 hours under nitrogen. Then solvent was removed and the residue was taken up in 20 mL dichloromethane, washed with 5% NaHCO₃ (100 mL) and saturated NaCl. The product was obtained after the evaporation of solvent as a pale yellow oil (868 mg, 95%). ¹H NMR (CDCl₃, 500 MHz) δ: 0.93(t, j-7.5 Hz, 9H), 1.68(q, j-7.5 Hz, 6H, 3.07(s, 6H), 3.22(s, 6H), 3.28(s, 2H), 3.50-3.62(m, 8H), 3.93(s, 18H), 4.54(s, 2H), 7.33(m, 5H); ¹³C NMR ((CDCl₃, 125 MHz δ: 7.39, 29.73, 35.16, 45.59, 69.24, 69.45, 69.51, 69.65, 70.07, 70.30, 70.54, 70.98, 73.16, 109.70, 127.54, 127.63, 128.27, 138.15 ppm.

### Example 12

### Protected pentaerythritol (Bn)(MOM)₃₍ 12)

Protected pentaerythritol (Bn) 3 (1.719 g, 7.6 mmol) was dissolved in dichloromethane (6 mL) and of diisopropylethyl amine (15 mL0 and was cooled to 0° C. Then methoxymethyl chloride (2.753 g, 34.2 mmol) was added dropwise. The reaction was stirred overnight. Then solvent was removed and the residue was taken up in 50 mL dichloromethane, washed with saturated sodium bicarbonate (4×100 mL) and brine. The product was purified by silica gel chromatography (ethyl acetate/hexane=1:6) to give 12 as a colorless oil (2.00 g, 73%). ¹H NMR (CDCl₃, 500 MHz) δ: 3.34(s, 9H), 3.53(s, 2H), 3.61(s, 6H, 4.51(s, 2H), 4.61(s, 6H), 7.25-7.37(m, 5H); ¹³C NMR (CDCl₃, 125 MHz) δ: 44.3, 54.9, 66.7, 69.1, 73.2, 96.7, 127.2, 127.2, 128.1, 138.6 ppm.

### Example 13

### Protected pentaerythritol (Bn)(MEM)₃ (13)

Protected pentaerythritol (BN) 3 (1.698 g, 7.5 mmol) was dissolved in dichloromethane (25 mL) and diisopropylethyl amine (5 mL). Then methoxyethoxymethylchloride (MEMCl, 3.083 g, 24.75 mmol) was added dropwise. The reaction was stirred for three hours. Then additional of MEMCl (1.12 g, total of 1.5 equiv./OH) was added. The reaction was stirred overnight. Then 10 mL dichloromethane was added and the mixture was washed with saturated sodium bicarbonate (3×50 mL). The crude was purified by silica gel chromatography (ethyl acetate/hexane=1:1) to give 13 as a colorless oil (2.56 g, 70%). ¹H NMR (CDCl₃, 500 MHz) δ: 3.40(s, 9H), 3.52(s, 2H), 3.53-3.56(m, 6H), 3.62(s, 6H), 3.66-3.68(m, 6H), 4.51(s, 2H), 4.71,(s, 6H), 7.25-7.37 3.34(s, 9H), 3.53(s, 2H), 3.61(s, 6H), 4.51(s, 2H), 4.61(s, 6H), 7.25-7.37(m, 5H), ¹³C NMR (CDCl₃, 125 MHz) δ: 44.3, 54.9, 66.7, 69.1, 73.2, 96.7, 127.2, 127.2, 128.1, 138.6 ppm.

### Example 14

### Protected pentaerythritol (OH)(OM)₃ (14)

Protected pentaerythritol (Bn)(MOM)₃ 12 (1.864 g, 5.20 mmol) was dissolved in 30 mL methanol. The mixture was purged with argon for 15 minutes. Then Pd/C (10% w/w of Pd on activated carbon, 400 mg) was added and the reaction was put on a Parr hydrogenator (55 psi) for 100 hours. The mixture was passed through a plug of Celite, after removal of methanol, the residue was passed through a plug of silica gel to remove trace of Pd/C to give the product as a colorless oil (1.19 g, 86.0%). ¹H NMR (CDCl₃. 500 MHz) δ:2.55(s, br, 1H), 3.34(s, 9H), 3.57 (s, 6H), 3.71(s, 2H),4.59(s, 6H); ¹³C NMR (CDCl₃, 125 MHz δ: 44.09, 55.21, 64.88, 67.92, 96.80 ppm.

### Example 15

### Protected Pentaerythritol (OH)MEM)₃ (15)

Following the Parr hydrogenation procedure, protected pentaeitol (Bn)MEM)₃ 13 (2.427 g, 4.95 mmol) was used and the product 15 was a colorless oil (1.847 g, 93.3%. ¹HNMR (CDCl₃, 500 MHz) δ: 2.81(s, br, 1H), 3.299s, 9H), 3.44-3.46(m, 6H), 3.47(s, 6h), 3.54(s, 2H), 3.56-3.58(m, 6H), 4.58(s, 6H); ¹³C NMR (CDCl₃, 125 MHz) δ: 44.21, 58.69, 63.13, 66/52, 67.10, 71.50, 95.48 ppm.

### Example 16

### Preparation of Bn-G1-(ethyl orthoester)₃ dendron (16)

Bn-G1-(ethyl orthoester)₃ 11 (470 mg, 0.602 mmol) was dissolved in 5 mL methanol, and concentrate HCl (0.12 mL) was added. The reaction was heated at 70° C. for 2 hours. After removal of solvent, the residue was put on high vacuum over night to give the deprotected dendron 16 9420 mg, 100%). This material was used for the next step reaction without further purification.

### Example 17

### Preparation of Bn-G1-(MOM)₉ (17)

Method 1. Diisopropylethyl amine (4.0 mL) and anhydrous dichloromethane (1.0 mL) was added to the flask containing Bn-G1-(OH)₉ polyether dendron 16 (402 mg, 0.601 mmol). This suspension was cooled to 0° C. using an ice-water bath. Then methoxymethyl chloride (1.31 g, 16.23 mmol) was added drop wise. After the addition the reaction was allowed to warm to room temperature and stirred overnight. Then solvent was removed and the residue was taken up in 10 mL dichloromethane and was washed with saturated sodium bicarbonate (4×20 mL) and brine. After silica gel purification the product is a colorless oil (245 mg, 38%).

### Example 18

### Preparation of Bn-G1-(MOM)₉ (17)

Method 2. A solution of Bn-G0-(Br)₃ 10 (551.6 mg, 1.10 mmol) in DMP (2 mL) was added to the alkoxide of 14 (1.058 g of 14 reacted with 133 mg of NaH in 2 mL of DMF). The reaction was heated at 100° C. for 12 hours. Then DMF was removed and the residue was taken up in 30 mL dichloromethane, washed with 5% sodium bicarbonate (3×50 mL) and brine. The crude was purified using silica gel chromatography to give the product as a colorless oil (388 mg, 46%). ¹H NMR (CDCl₃, 500 MHz) δ: 3.32(s, 27H), 3.35(s, 6H), 3.36(s, 6H), 3.42(s, 2), 3.51(s, 18H), 3.52-3.53(m, 2), 3.55-3.57(m, 2H), 3.59-3.60(m, 2H), 3.68-3.70(m, 2H), 4.59(s, 18H), ¹³C NMR (CDCl₃, 125 MHz) δ: 44.50, 45.91, 55.00, 61.81, 66.93, 70.08, 70.43, 70.51, 70.53, 71.11, 72.50, 96.80 ppm.

### Example 19

### Preparation of Gold Nanoparticles

General Preparation

1. Prepare 1 mL of a 4% HAuCl₄ solution in deionized water.

2. Add 375 microliters of the chloroauric acid solution plus 500 microliters of 0.2 M potassium carbonate to 100 mL deionized water, cool on ice to 4° C. and mix well.

3. Dissolve sodium borohydride in 5 mL of water at a concentration of 0.5 mg/mL and prepare fresh.

4. Add five 1 mL aliquots of the sodium borohydride solution to the chloroauric acid/carbonate suspension with rapid stirring. A color change fro bluish-purple to reddish-orange will be noted as the addition takes place.

5. Stir for 5 min. on ice after the completion of the sodium borohydride addition.

### Example 20

### Preparation of the Dendron

Dendrimers containing cystamine cores were reduced using dithiothreitol (DTT) to yield single site, thiol core, functionalized PAMAM dendron reagents.

Cystamine core, carboxylic acid surface dendrimer (0.0254 mmol) was dissolved in deionized water (0.5 mL, purged with nitrogen for 15 minutes.) Then DTT soluti9on (0.9 eq. per disulfide) was added. The reaction was stirred overnight under nitrogen. TLC check showed there was no free DTT left and the dendrimer was reduced.

In a 250 mL round bottomed flask was place 100 mL deionized water and a magnetic stir bar and the flask was cooled to 4° C. with an ice/water bath. About 500 microliters of 0.2 M potassium carbonate solution and 375 microliters of 4% HAuCl₄ was added and mixed well. Then 5 mL NaBH₄ solution was added, 1 mL at a time with rapid stirring. A color change from blusih-purple to reddish-orange was noted as the addition takes place. The reaction was stirred for 5 more minutes under this temperature. Then the dendron solution was added quickly, the color of the reaction became darker. The reaction was stirred at 4° C. for another 10 minutes and then ice/water bath was removed. The reaction was then allowed to warm to room temperature and stirred overnight under dark. Then water was removed under reduced pressure at room temperature water bath. For Au-G1-COOH. Methanol (4 mL) was added to the purple reside and a black precipitate was obtained. The methanol layer was clear. The black precipitate was washed with methanol three more times to remove any excess of dendrimer. For Au-G2-COOH and Au-G3-COOH, the residue was redissolved in 0.5 mL of water, and purified through Sephadex G50 for G2 and Sephadex G100 for G3 columns respectively, to remove excess dendrimer. TEM images of G1, G2, and G3 dendron coated gold nanoparticle were then obtained.

### Example 21

### Polyether Dendron with Phosphine at the Focal Point

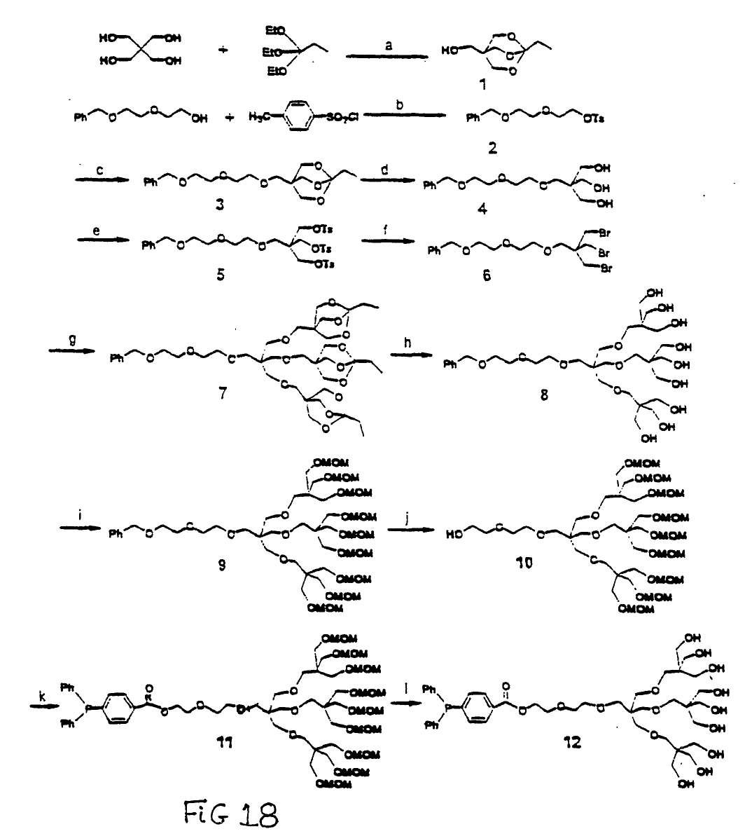

With reference to FIG. 18, the design of the dendron ligand is based on the following. Aryl phosphine is used as a focal point binding site to the quantum dot because of its stability in air and it is less toxic than alky phosphines. The aryl groups, which are UV active at 200 nm will not block any photoluminescence, that is above 500 nm. Most importantly, phosphine passivation may not quench the PL which is essential for bio-labeling. The two units of ethylene diglycol chain between the focal point and the dendritic structure are for enhancement of aqueous solubility. Pentaerytritol was used as the AB₃ branching unit because it can reach a more close packing point than AB₂ while generating growth, which can provide a dense packing at a lower generation. The surface functional groups are methoxymethyl ether protected hydroxyls that can be deprotected to release nine hydroxyls, so it can be either hydrophobic or hydrophilic, and hydroxyl groups can be subjected to further modifications. The synthesis of the dendritic polyether phosphine ligands to generation 2 are shown in FIG. 18. In FIG. 18, (a) is pyridinium p-toluenesulfonate, at 130° C.; (b) is pyridine, −12° C.; (c) is NaH, 1, DMF, 100° C.; (d) is trace of HCl, MeOH; (e) is TsCl, Pyridine, room temperature; (f) is NaBr, DMAc, 130° C.; (g) is NaH, 1, DMF, 100° C.; (h) is trace HCl, MeOH; (i) is MOMCl, diisopropylethylamine/CH₂Cl₂; (j) is H₂/Palladium on carbon, MeOH; (k) is 4-(diphenylphosphino)benzoic acid, DCC, DMAP. CH₂Cl₂; (l) is 0.1MHCl. MeOH, 40° C. The water-soluble citrate stabilized core-shell CdSe/CdS quantum dots were made using previously reported methods.

The luminescence has a sharp {full width at half maximum (fwhm)) 36 nm}, symmetrical emission at 563 nm which is indicative of a 3.5 nm CdSe core. The core-shell quantum dots showed a narrow size distribution with no detectable surface trap emission. (see FIGS. 16 and 17 wherein (i) is the citrate stabilized dots, (ii) is the Generation −2 polyether phosphine ligand 12 and (iii) is the Generation −2 PAMAM sulfhydryl ligand.

What is claimed is:

1. A method of stabilizing nanoparticles selected from the group consisting of semiconductor nanoparticles, metal nanoparticles and metal salt nanoparticles, the method comprising contacting dendrons containing single focal point functional groups, with colloidal solutions selected from the group of colloidal solutions consisting of semiconductor, metal, and metal salt nanoparticles and allowing the single focal point functional groups to react with the surfaces of the semiconductor, metal, and metal salt nanoparticles to obtain stabilized, dendronized, semiconductor, metal, and metal salt nanoparticles.

2. A method of stabilizing nanoparticles selected from the group consisting of semiconductor nanoparticles, metal nanoparticles, and metal salt nanoparticles, the method comprising contacting organic dendrons containing single focal point sulfhydryl groups, with colloidal solutions of semiconductor, metal, and metal salt nanoparticles and allowing the single focal point sulfhydryl groups to react with the surfaces of the semiconductor, metal, and metal salt nanoparticles to obtain stabilized, dendronized, semiconductor, metal, and metal salt nanoparticles.

3. A method of stabilizing semiconductor, metal, and metal salt nanoparticles, the method comprising contacting organic dendrons containing single focal point phosphine groups, with colloidal solutions of semiconductor, metal, and metal salt nanoparticles and allowing the single focal point phosphine groups to react with the surfaces of the semiconductor, metal, and metal salt nanoparticles to obtain stabilized, dendronized, semiconductor, metal, and metal salt nanoparticles.

4. A method of stabilizing nanoparticles selected from the group consisting of semiconductor, metal, and metal salt nanoparticles, the method comprising contacting organic dendrons containing single focal point phosphine oxide groups, with colloidal solutions of semiconductor, metal, and metal salt nanoparticles and allowing the single focal point phosphine oxide groups to react with the surfaces of the semiconductor, metal, and metal salt nanoparticles to obtain stabilized, dendronized, semiconductor, metal, and metal salt nanoparticles.

5. A method according to claim 1 wherein the semiconductor, metal, and metal salt nanoparticles are passivated prior to contacting them with the single focal point functional groups.

5. A method according to claim 2 wherein the semiconductor, metal, and metal salt nanoparticles are passivated prior to contacting them with the single focal point functional groups.

6. A method according to claim 3 wherein the semiconductor, metal, and metal salt nanoparticles are passivated prior to contacting them with the single focal point functional groups.

7. A method according to claim 4 wherein the semiconductor, metal, and metal salt nanoparticles are passivated prior to contacting them with the single focal point functional groups.

8. A method according to claim 1 wherein the outside surfaces of the dendrons contain functional groups.

9. A method according to claim 2 wherein the outside surfaces of the dendrons contain functional groups.

10. A method according to claim 3 wherein the outside surfaces of the dendrons contain functional groups.

11. A method according to claim 4 wherein the outside surfaces of the dendrons contain functional groups.

12. A method according to claim 5 wherein the outside surfaces of the dendrons contain functional groups.

13. A method as claimed in claim 8 wherein the functional groups on the outside surfaces of the dendrons are selected from the group consisting of: (i) hydrophilic groups, (ii) hydrophobic groups, (iii) reactive groups, and (iv) passive groups.

14. A method as claimed in claim 8 wherein the functional groups on the outside surfaces of the dendrons are selected from the group consisting of: (i) hydrophilic groups, (ii) hydrophobic groups, (iii) reactive groups, and (iv) passive groups.

15. A method as claimed in claim 8 wherein the functional groups on the outside surfaces of the dendrons are selected from the group consisting of. (i) hydrophilic groups, (ii) hydrophobic groups, (iii) reactive groups, and (iv) passive groups.

16. A method as claimed in claim 8 wherein the functional groups on the outside surfaces of the dendrons are selected from the group consisting of: (i) hydrophilic groups, (ii) hydrophobic groups, (iii) reactive groups, and (iv) passive groups.

17. A method as claimed in claim 8 wherein the functional groups on the outside surfaces of the dendrons are selected from the group consisting of. (i) hydrophilic groups, (ii) hydrophobic groups, (iii) reactive groups, and (iv) passive groups.

18. A method as claimed in claim 13 wherein the reactive groups are selected from the group consisting of: hydroxyl, amino, carboxylic, sulfonic, sulfonato, mercapto, amido, phosphino, —NH—COPh, —COONa, alkyl, aryl, ester, heterocylic, alkynyl, and alkenyl.

19. A method as claimed in claim 3 wherein the phosphine group has the formula: 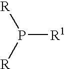
wherein each R is independently selected from alkyl radicals having 1 to 4 carbon atoms and aryl groups, and R¹ is a functionally reactive connector group.

20. A method as claimed in claim 4 wherein the phosphine group has the formula: 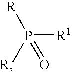
wherein each R is independently selected from alkyl radicals having 1 to 4 carbon atoms and aryl groups, and R¹ is a functionally reactive connector group

21. A method of stabilizing nanoparticles selected from the group consisting of semiconductor nanoparticles, metal nanoparticles, and metal salt nanoparticles, the method comprising contacting organic dendrons containing single focal point sulfhydryl groups, with colloidal solutions of semiconductor, metal, and metal salt nanoparticles and allowing the single focal point sulfhydryl groups to react with the surfaces of the semiconductor, metal, and metal salt nanoparticles to obtain stabilized, dendronized, semiconductor, metal, and metal salt nanoparticles, wherein the single focal point sulfhydryl group containing dendron is prepared by the method comprising:
(I) providing a dendrimer have a disulfide core;
(II) reducing the disulfide of the disulfide core dendrimer to form sulfhydryl functional dendrons;
(III) contacting the sulfhydryl functional dendrons with a colloidal solution of nanoparticles to obtain dendronized semiconductor, metal, and metal salt nanoparticles.

22. The method as claimed in claim 21 wherein the semiconductor, metal, and metal salt nanoparticles cores are selected from any metal that can be made into a colloidal solution.

23. A method as claimed in claim 19 wherein the functionally reactive connector group contains at least one ethylene oxide unit.

24. A method as claimed in claim 23 wherein the connector group has from 1 to 10 ethylene oxide units.

25. A method as claimed in claim 20 wherein the functionally reactive connector group contains at least one ethylene oxide unit.

26. A method as claimed in claim 25 wherein the connector group has from 1 to 10 ethylene oxide units.

27. A composition of matter, said composition of matter being colloidal solutions selected from the group consisting of semiconductor nanoparticles, metal nanoparticles, and metal salt nanoparticles having outside surfaces, said outside surfaces having attached thereto, dendrons, said attachment comprising a linking group selected from the group consisting of:
(i) sulfur as the thiol,
(ii) thiol in combination with ethylene oxide units, and
(iii) phosphorus, wherein the phosphorus is in the form of a group selected from
(a) phosphines, and
(b) phosphine oxides in combination with ethylene oxide units.

28. A composition of matter as claimed in claim 27 wherein (a) in combination with ethylene oxide has the general formula: 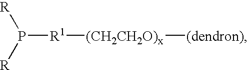
whereinₓ has a value of from 1 to 10, each R is independently selected from alkyl groups of 1 to 4 carbon atoms and aryl groups and R¹ is a connector group.

29. A composition of matter as claimed in claim 27 wherein (b) in combination with ethylene oxide has the general formula: 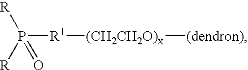
whereinₓ has a value of from 1 to 10, wherein each R is independently selected from alkyl groups of 1 to 4 carbon atoms and aryl groups and R¹ is a connector group.

30. A composition of matter as claimed in claim 27 wherein (ii) in combination with ethylene oxide has the general formula: HSR¹—(CH₂CH₂O)ₓ— (dendron), whereinₓ has a value of from 1 to 10, wherein R¹ is a connector group.

31. A composition of matter as claimed in claim 27 wherein the nanoparticle core is iron.

32. A composition of matter as claimed in claim 27 wherein the nanoparticle core is gold.

33. A composition of matter as claimed in claim 27 wherein the nanoparticle core is copper.

34. A composition of matter as claimed in claim 27 wherein the nanoparticle core is platinum.

35. A composition of matter as claimed in claim 27 wherein the nanoparticle core is palladium.

36. A composition of matter as claimed in claim 27 wherein the nanoparticle core is cobalt.

37. A composition of matter as claimed in claim 27 wherein the nanoparticle core is nickel.

38. A composition of matter as claimed in claim 27 wherein the nanoparticle core is zinc.

39. A composition of matter as claimed in claim 27 wherein the nanoparticle core is cadmium.

40. A composition of matter as claimed in claim 27 wherein the nanoparticle core is iron oxide.

41. A composition of matter as claimed in claim 27 wherein the nanoparticle core is CdSe.

42. A composition of matter as claimed in claim 27 wherein the nanoparticle core is CdS.

43. A composition of matter as claimed in claim 27 wherein the nanoparticle core is CdSe/CdS.

44. A composition of matter as claimed in claim 27 wherein the nanoparticle core is CdSe/ZnS.

45. A composition of matter as claimed in claim 27 wherein the nanoparticle core is CdTe.

46. A composition of matter as claimed in claim 27 wherein the nanoparticle core is CdTe/CdS.

47. A composition of matter as claimed in claim 27 wherein the nanoparticle core is CdTe/ZnS.

48. The use of the composition of matter of claim 31 as an MRI agent.

49. The use of the composition of matter of claim 32 as a projectile for a gene gun.

50. The use of a composition of claim 1 wherein the use is selected from the group consisting of: biologically active materials, genetic materials, biologically active materials for use as vaccines, biomedical tags, components in light emitting diode devices, diagnostics, nanosensors, nano-arrays for DNA and RNA, protein applications, chelators, photon absorption, energy absorbing, energy emitting, signal generator for diagnostics, and radioactive materials.
